(19) United States
(12) Patent Application Publication
(10) Pub. No.: US 2023/0293641 A1
(43) Pub. Date: Sep. 21, 2023

(54) COMPOSITIONS AND METHODS FOR REDUCING URIC ACID CONCENTRATION USING NANOCAPSULE-BASED DRUG DELIVERY SYSTEM

(71) Applicant: VL Biotech Inc., New Taipei (TW)

(72) Inventors: Hsin-Yi LIN, New Taipei (TW); Stanley KUNG, New Taipei (TW)

(21) Appl. No.: 18/185,231

(22) Filed: Mar. 16, 2023

Related U.S. Application Data
(60) Provisional application No. 63/320,819, filed on Mar. 17, 2022.

(51) Int. Cl. A61K 38/44; A61K 48/00; A61K 9/51; C12N 15/70

(52) U.S. Cl. CPC A61K 38/44; A61K 48/0041; A61K 9/51; C12Y 305/01032; C12N 15/70; C12N 2800/101

(57) ABSTRACT

Disclosed herein are compositions and methods with enhanced capability to reduce uric acid concentration. The compositions are loaded in a nanocarrier for oral drug delivery. The composition comprises a first vector and a second vector. The first vector encodes one or more mRNAs. The one or more mRNAs encode a peptide with uricase activity and are labeled with one or more capsid protein tags. The second vector encodes one or more capsid proteins. The one or more capsid proteins bind to the one or more capsid protein tags on the one or more mRNA.

### CROSS REFERENCE TO RELATED APPLICATIONS

This application claims priority of U.S. Provisional Application No. 63/320,819 filed on Mar. 17, 2022 under 35 U.S.C. § 119(e), the entire contents of all of which are hereby incorporated by reference.

### FIELD OF THE INVENTION

The present invention relates to compositions and methods of use, in particular to therapeutic compositions with enhanced capability to reduce uric acid concentration in a human or animal subject, which are delivered by using nanocapsule-based drug delivery system. Compositions and methods of the present invention are particularly applicable to a range of disorders, including gout, hyperuricemia and related conditions.

### BACKGROUND OF THE INVENTION

Uric acid is the product of the metabolism of purine. Under normal circumstances, uric acid in the human body is mainly excreted in urine through kidney filtration, and a small amount is catabolized by intestinal bacteria. However, due to the lack of uricase in the human body that is able to directly decompose uric acid, once uric acid is overproduced or metabolized abnormally, the concentration of uric acid in the blood will continue to rise, and then uric acid crystals will form in the body and accumulate in joints or in other tissues, it causes diseases such as gout or kidney stones.

Although urate oxidase synthesized by genetic recombination is currently used as a drug for the treatment of gout, it mimics the function of uricase to decompose uric acid into highly soluble allantoin, but this urate oxidase is an exogenous protein to the human body, easily cause side effects such as allergic reaction or hemolysis.

### SUMMARY OF THE INVENTION

The present disclosure provides a composition for controlling uric acid. The composition comprises a first vector and a second vector. The first vector comprises the nucleotide sequence as denoted by SEQ ID NO: 1 and a capsid protein recognition sequence. The first vector encodes one or more mRNAs encoding a peptide with uricase activity and the one or more mRNAs bind to one or more capsid protein tags. The second vector comprises one or more nucleotide sequences encoding one or more capsid proteins. The one or more capsid proteins are capable of self-assembling into one or more virus-like particles that encapsulate the one or more mRNAs such that the composition is fabricated as nanocapsule.

The present disclosure also provides a composition for controlling uric acid. The composition comprises a first vector and a second vector. The first vector comprises the nucleotide sequence as denoted by SEQ ID NO: 5. The second vector comprises the nucleotide sequence as denoted by SEQ ID NO: 3. The nucleotide sequence of SEQ ID NO: 3 encodes one or more capsid proteins, and the one or more capsid proteins are capable of self-assembling into one or more virus-like particles that encapsulate one or more mRNAs encoded by the nucleotide sequence of SEQ ID NO: 5 such that the composition is fabricated as nanocapsule.

The present disclosure further provides a method for controlling uric acid concentration in a subject. The method includes administering a composition comprising one or more nanocapsules and/or encapsulated microbial cells to the subject. The one or more nanocapsules and/or encapsulated microbial cells are transformed by a first vector and a second vector. The first vector encodes one or more mRNAs, and the one or more mRNAs encode a peptide with uricase activity. The one or more mRNAs are labeled with one or more capsid protein tags. The second vector comprises one or more nucleotide sequences encoding one or more capsid proteins. The one or more capsid proteins are capable of self-assembling into one or more virus-like particles that encapsulate the one or more mRNAs such that the composition is fabricated as the one or more nanocapsules or/and encapsulated microbial cells.

### BRIEF DESCRIPTION OF THE DRAWINGS

FIG. 1 illustrates a western blot showing the recognition of uricase production via a non-uricase-capable cell line.

FIG. 2 illustrates plots demonstrating the uric acid content of the test hamster with hyperuricemia significantly decreased after administration of the Composition 1.

### DETAILED DESCRIPTION OF THE PREFERRED EMBODIMENTS

As used herein, the term “vector” used in reference to delivery of components refers to a nucleic acid capable of transporting between different genetic environments another nucleic acid to which it has been operatively linked. The term “express” in the context of the disclosure refers to transcription and/or translation of a specific nucleotide sequence driven by its promoter.

The disclosure relates to one or more nucleotide sequences encoding a peptide with uricase activity, which particularly can be assembled into nanocapsule in bacteria (such as probiotics), for reducing uric acid concentration. Thus, the one or more nucleotide sequences can be incorporated into pharmaceutical and/or therapeutic compositions suitable for administration.

The present disclosure discloses a first vector and a second vector, the first vector encodes one or more messenger ribonucleic acids (messenger RNAs, mRNAs) encoding a peptide with uricase activity. The one or more mRNAs are further labeled with one or more capsid protein tags. The second vector encodes one or more capsid proteins (CPs), which may be self-assembled to form one or more hollow virus-like particles (virus-like particles, VLPs), then bind to and provide domain specificity to the one or more capsid protein tags on the one or more mRNAs, such that the one or more mRNAs are capable of being encapsulated in the one or more VLPs. Thus, the one or more mRNAs encoding the peptide with uricase activity are carried in nanocarrier and will be delivered via nanocapsules after oral administration.

In some aspects, the pharmaceutical and/or therapeutic compositions comprise bio-products or compounds transcribed or translated from the first vector and the second vector described in the present disclosure.

The pharmaceutical and/or therapeutic composition may be used to alleviate a disorder of uric acid metabolism. Therefore, in one aspect, it is contemplated that the compositions and methods of the disclosure are used to treat and/or prevent disorders of uric acid metabolism, such as gout, hyperuricemia, uric acid nephrolithiasis, recurrent acute gouty arthritis, chronic gouty arthritis, joint deformities, uric acid nephropathy, and combinations thereof.

In one aspect, the disclosure relates to one or more nanocapsule containing bacteria, which is assembled from one or more bio-products transcribed or translated from the first vector and the second vector. The bio-products may be either the mRNAs or the capsid proteins.

In another aspect, the disclosure relates to one or more nanocapsule assembled from one or more bio-products transcribed or translated from the first vector and the second vector. The bio-products may be either the mRNAs or the capsid proteins.

In yet another aspect, the disclosure relates to one or more VLPs assembled from one or more bio-products transcribed or translated from the first vector and the second vector. The bio-products may be either the mRNAs or the capsid proteins.

In further aspect, the disclosure provides a use of the pharmaceutical and/or therapeutic composition of the disclosure in a preparation of a medicament for a treatment of disorders of uric acid metabolism, such as gout, hyperuricemia, uric acid nephrolithiasis, recurrent acute gouty arthritis, chronic gouty arthritis, joint deformities, uric acid nephropathy, and combinations thereof.

In another aspect, the disclosure provides a method for treating and/or preventing disorders of uric acid metabolism, such as hyperuricemia or gout, which comprises administering the pharmaceutical and/or therapeutic composition of the disclosure or the preparation thereof.

In one embodiment, the first vector comprises the nucleotide sequence as denoted by SEQ ID NO: 1, and a capsid protein recognition sequence. The first vector is transcribed to form mRNA with the one or more capsid protein tags, which encodes a peptide with uricase activity and can be used as a translation template in a cell of a human or animal subject to produce a peptide with uricase activity in the human or animal subject's body, to endogenously produce uricase in vivo. In addition, the one or more capsid protein tags can bind to a specific region of one or more capsid proteins because of a secondary structure formed by the capsid protein recognition sequence. In one embodiment, the capsid protein recognition sequence comprises the nucleotide sequence as denoted by SEQ ID NO: 2, and the one or more capsid protein tags formed by the capsid protein recognition sequence has a hairpin structure.

Further, the second vector comprises one or more nucleotide sequences encoding the one or more capsid proteins. In one embodiment, the one or more capsid proteins are shell proteins derived from bacteriophages, such as AP205 phage, Qbeta phage, MS2 phage, P22 phage and the like. In another embodiment, the second vector comprises the nucleotide sequence as denoted by SEQ ID NO: 3, and the one or more capsid proteins encoded by the nucleotide sequence of SEQ ID NO: 3 can be bound with the one or more capsid protein tags formed by the nucleotide sequence of SEQ ID NO: 2.

The first vector of the composition expresses the one or more mRNAs encoding the peptide with uricase and the second vector of the composition expresses the one or more capsid proteins. The one or more capsid protein tags on the one or more mRNAs facilitate the combination of the capsid proteins and the one or more mRNAs. Consequently, the one or more capsid proteins are self-assembled into the one or more virus-like particles which can be utilized to deliver the one or more RNAs encoding the peptide with uricase activity in the form of nanocapsules.

The first vector may further comprise an internal ribosome entry site (IRES) containing the nucleotide sequence as denoted by SEQ ID NO: 4. The internal ribosome entry site is placed between the nucleotide sequence of SEQ ID NO: 1 and the capsid protein recognition sequence. The first vector may include a promoter and a terminator for RNA polymerase to perform transcription, for example, a P11 promoter and a T1dh terminator may be used, but not limited to this.

In another embodiment, the composition of the disclosure comprises a first vector and a second vector. The first vector comprises the nucleotide sequence as denoted by SEQ ID NO: 5 encoding one or more capsid protein tags and one or more mRNAs which encodes a peptide with uricase activity. The second vector comprises the nucleotide sequence as denoted by SEQ ID NO: 3 encoding one or more capsid proteins. The one or more capsid proteins encoded by the nucleotide sequence of SEQ ID NO: 3 can be combined with the one or more capsid protein tags formed by the nucleotide sequence of SEQ ID NO: 5.

The disclosure further provides a nanocapsule-based drug delivery system for delivering mRNA into the body of the human or animal subject to reduce uric acid concentration of the human or animal subject. In one embodiment, the nanocapsule-based drug delivery system is achieved by various administrations, which are used to enable delivery of the compositions to the desired site of biological action. These administrations include, but are not limited to oral routes, intraduodenal routes, parenteral injection (including intravenous, subcutaneous, intraperitoneal, intramuscular, intravascular or infusion), topical and rectal administration.

In one embodiment, the first vector and the second vector are transformed into a transformed cell, and the genetic information of the first vector and the second vector are expressed through the physiological mechanism of the transformed cell. In an example, the transformed cells are bacterial cells. In the process of the transformation (or the bacterial cells are produced), the first vector and the second vector are either transcribed into mRNAs or translated into capsid proteins; the bio-products are then self-assembled into the VLPs, encapsulated in the bacterial cells. As the VLPs are nano-scale particles, the one or more nucleotide sequences are assembled into nanocapsule containing bacterial cells. In an alternative example, the transformed cells are probiotics. In the process of the transformation (or the probiotics are produced), the first vector and the second vector are either transcribed into mRNAs or translated into capsid proteins; the bio-products are then self-assembled into the VLPs, encapsulated in the probiotics. As the VLPs are nano-scale particles, the one or more nucleotide sequences are assembled into nanocapsule containing probiotics.

Specifically, the first vector is expressed by the transformed cell to produce the one or more mRNAs with the one or more capsid protein tags, and the one or more mRNAs encode a peptide with uricase activity in the body of the human or animal subject. The second vector is expressed by the transformed cell to form one or more capsid proteins. The products of the first vector and the second vector can be self-assembled into nanocapsule in the transformed cell by binding of the one or more capsid protein tags to the one or more capsid proteins.

The composition of the disclosure can promote the body of the human or animal subject to produce the peptides with uricase activity, which are more similar to endogenous uricase than artificially synthesized uricase derivatives. The uricase produced in vivo is effectively capable of removing excess urate in the blood of the human or animal subject, so as to enable therapeutic effects by enzymatic lowering uric acid concentration in the blood.

In one embodiment, the transformed cells are bacterial cells, which can be easily obtained and cultured from bacterial cultures. The bacterial cells may be Escherichia coli.

The following examples are disclosed to demonstrate that the composition of the disclosure is able to endogenously produce uricase in vivo, causing a decrease in uric acid concentration or maintaining uric acid concentration at pretreatment levels. The following examples are only for the purpose of illustration, and the scope of the disclosure is not limited by the examples. Those skilled in the art can utilize the disclosure and teachings of the disclosure to create other embodiments, aspects and variations without undue experimentation.

### Example 1

The Composition 1 is prepared by transforming the first vector and the second vector into Escherichia coli cells. The first vector adopts pT7CFE1-NHA vector and contains the nucleotide sequence of SEQ ID NO: 5. The second vector adopts pCDF-1b vector and contains the nucleotide sequence of SEQ ID NO: 3. Then the Composition 1 is administered to HeLa cells by adding into the culture medium of Hela cells and cultured for 12 hours.

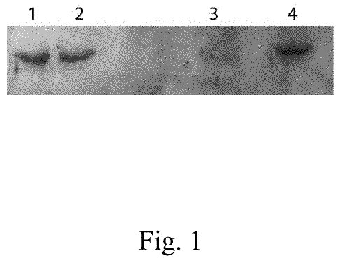

The western blot method is carried out to analyze whether the culture medium after 12 hours of cultivation contains peptides with uricase activity. In FIG. 1, the first flow channel and the second flow channel are filled with commercially available uricase, the third channel is filled with the culture medium of HeLa cells without administering the Composition 1 as a control group, and the fourth channel is filled with the culture medium of HeLa cells administered the Composition 1. As shown in FIG. 1, it can be seen that HeLa cells inherently without uricase activity will produce uricase after being cultured with the Composition 1 for 12 hours, which demonstrates that the Composition 1 can promote eukaryotic cells to produce uricase.

### Example 2

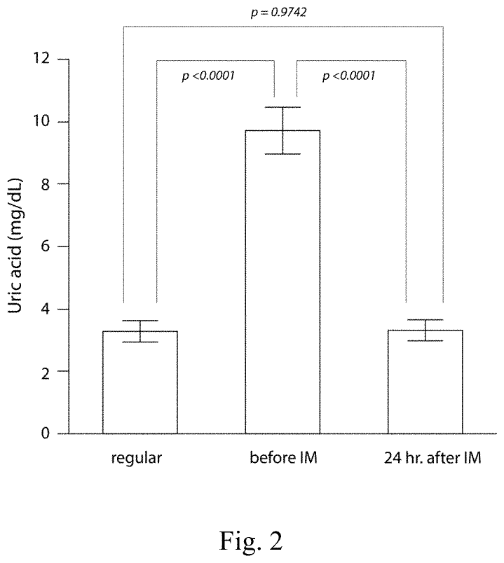

Hamster was used to demonstrate the effect of lowering uric acid levels in the blood. The test hamster received hypoxanthine and potassium oxonate (PO) to induce hyperuricemia. Further, the Composition 1 is injected intramuscularly (IM) into the test hamster. After 12 hours of injection, the test hamster's blood was collected to measure the uric acid content in the serum. The results were shown in FIG. 2.

It can be clearly seen from FIG. 2 that the uric acid content of the test hamster with hyperuricemia significantly decreased after administration of the Composition 1, and the uric acid content even reached a normal level. In summary, the Composition 1 directly decreases blood uric acid level in vivo, hence being expected to be useful for alleviating disorders of uric acid metabolism.

What is claimed is:

1. A composition for reducing uric acid concentration, comprising:
a first vector, comprising the nucleotide sequence as denoted by SEQ ID NO: 1 and a capsid protein recognition sequence, wherein the first vector encodes one or more mRNAs encoding a peptide with uricase activity and the one or more mRNAs are labeled with one or more capsid protein tags; and
a second vector, comprising one or more nucleotide sequences encoding one or more capsid proteins, wherein the one or more capsid proteins are capable of self-assembling into one or more virus-like particles that encapsulate the one or more mRNAs such that the composition is fabricated as nanocapsule.

2. The composition according to claim 1, wherein the capsid protein recognition sequence in the first vector comprises the nucleotide sequence as denoted by SEQ ID NO: 2, and the second vector comprises the nucleotide sequence as denoted by SEQ ID NO: 3.

3. The composition according to claim 1, wherein the first vector further comprises an internal ribosome entry site (IRES) between the nucleotide sequence of SEQ ID NO: 1 and the capsid protein recognition sequence.

4. A composition for reducing uric acid concentration, comprising:
a first vector, comprising the nucleotide sequence as denoted by SEQ ID NO: 5; and
a second vector, comprising the nucleotide sequence as denoted by SEQ ID NO: 3;
wherein the nucleotide sequence of SEQ ID NO: 3 encodes one or more capsid proteins, and the one or more capsid proteins are capable of self-assembling into one or more virus-like particles that encapsulate one or more mRNAs encoded by the nucleotide sequence of SEQ ID NO: 5 such that the composition is fabricated as nanocapsule.

5. A method for reducing uric acid concentration in a subject, comprising:
administering a composition comprising one or more nanocapsules and/or encapsulated microbial cells to the subject, wherein the one or more nanocapsules or/and encapsulated microbial cells are transformed by a first vector and a second vector, the first vector encoding one or more mRNAs, wherein the one or more mRNAs encode a peptide with uricase activity, and the one or more mRNAs are labeled with one or more capsid protein tags, the second vector comprising one or more nucleotide sequences encoding one or more capsid proteins, wherein the one or more capsid proteins are capable of self-assembling into one or more virus-like particles that encapsulate the one or more mRNAs such that the composition is fabricated as the one or more nanocapsules and/or encapsulated microbial cells.

6. The method according to claim 5, wherein the first vector comprises the nucleotide sequence as denoted by SEQ ID NO: 1 and a capsid protein recognition sequence.

7. The method according to claim 5, wherein the capsid protein recognition sequence in the first vector comprises the nucleotide sequence as denoted by SEQ ID NO: 2, and the second vector comprises the nucleotide sequence as denoted by SEQ ID NO: 3.

8. The method according to claim 5, wherein the first vector further comprises an internal ribosome entry site (IRES) between the nucleotide sequence of SEQ ID NO: 1 and the capsid protein recognition sequence.

9. The method according to claim 5, wherein the first vector comprises the nucleotide sequence as denoted by SEQ ID NO: 5, and the second vector comprises the nucleotide sequence as denoted by SEQ ID NO: 3.


SEQUENCE LISTING (5 sequences)

SEQ ID NO: 1 - DNA, 936 bp, synthetic construct
  source 1..936: mol_type = other DNA
gcaaagggaaatggctcattaccgtaatgactacaaaaagaatgatgaggtagagtttgtccgaactggctatgggaaggatatgataaaagttctccatattcagcgagatggaaaatatcacagcattaaagaggtggcaacttcagtgcaactgactttgagctccaaaaaagattacctgcatggagacaattcagatgtcatccctacagacaccatcaagaacacagttaatgtcctggcgaagttcaaaggcatcaaaagcatagaaacttttgctgtgactatctgtgagcatttcctttcttccttcaagcatgtcatcagagctcaagtctatgtggaagaagttccttggaagcgttttgaaaagaatggagttaagcatgtccatgcatttatttatactcctactggaacgcacttctgtgaggttgaacagataaggaatggacctccagtcattcattctggaatcaaagacctaaaagtcttgaaaacaacccagtctggctttgaaggattcatcaaggaccagttcaccaccctccctgaggtgaaggaccggtgctttgccacccaagtgtactgcaaatggcgctaccaccagggcagagatgtggactttgaggccacctgggacactgttaggagcattgtcctgcagaaatttgctgggccctatgacaaaggcgagtactcgccctctgtccagaagacactctatgacatccaggtgctcaccctgggccaggttcctgagatagaagatatggaaatcagcctgccaaatattcactacttaaacatagacatgtccaaaatgggactgatcaacaaggaagaggtcttgctacctttagacaatccatatggcaggattactggtacagtcaagaggaagctgacttcaaggctgtgatgttatggccc

SEQ ID NO: 2 - DNA, 29 bp, synthetic construct
  source 1..29: mol_type = other DNA
gggaatcacatcagtaactgagtgacttc

SEQ ID NO: 3 - DNA, 402 bp, synthetic construct
  source 1..402: mol_type = other DNA
atggcaaaattagagactgttactttaggtaacatcgggaaagatggaaaacaaactctggtcctcaatccgcgtggggtaaatcccactaacggcgttgcctcgctttcacaagcgggtgcagttcctgcgctggagaagcgtgttaccgtttcggtatctcagccttctcgcaatcgtaagaactacaaggtccaggttaagatccagaacccgaccgcttgcactgcaaacggttcttgtgacccatccgttactcgccaggcatatgctgacgtgaccttttcgttcacgcagtatagtaccgatgaggaacgagcttttgttcgtacagagcttgctgctctgctcgctagtcctctgctgatcgatgctattgatcagctgaacccagcgtattga

SEQ ID NO: 4 - DNA, 517 bp, synthetic construct
  source 1..517: mol_type = other DNA
ggcgaattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatg

SEQ ID NO: 5 - DNA, 1678 bp, synthetic construct
  source 1..1678: mol_type = other DNA
taatacgactcactataggggaatcacatcagtaactgagtgacttcggcgaattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggctcattaccgtaatgactacaaaaagaatgatgaggtagagtttgtccgaactggctatgggaaggatatgataaaagttctccatattcagcgagatggaaaatatcacagcattaaagaggtggcaacttcagtgcaactgactttgagctccaaaaaagattacctgcatggagacaattcagatgtcatccctacagacaccatcaagaacacagttaatgtcctggcgaagttcaaaggcatcaaaagcatagaaacttttgctgtgactatctgtgagcatttcctttcttccttcaagcatgtcatcagagctcaagtctatgtggaagaagttccttggaagcgttttgaaaagaatggagttaagcatgtccatgcatttatttatactcctactggaacgcacttctgtgaggttgaacagataaggaatggacctccagtcattcattctggaatcaaagacctaaaagtcttgaaaacaacccagtctggctttgaaggattcatcaaggaccagttcaccaccctccctgaggtgaaggaccggtgctttgccacccaagtgtactgcaaatggcgctaccaccagggcagagatgtggactttgaggccacctgggacactgttaggagcattgtcctgcagaaatttgctgggccctatgacaaaggcgagtactcgccctctgtccagaagacactctatgacatccaggtgctcaccctgggccaggttcctgagatagaagatatggaaatcagcctgccaaatattcactacttaaacatagacatgtccaaaatgggactgatcaacaaggaagaggtcttgctacctttagacaatccatatggcaggattactggtacagtcaagaggaagctgacttcaaggctgtgagaattcgatatcttaattaagctgcaggagctcgtcgacgcggccgcactcgagtgagatctgactgaaaaaaaaaaaaaaaaaaaaaaaaaaaaaagtttaaacgggaatcacatcagtaactgagtgacttcactagtccgctgagcaataactagcataaccccttggggcctctaaacgggtcttgaggggttttttg